(19) United States
(12) Patent Application Publication
(10) Pub. No.: US 2023/0109918 A1
(43) Pub. Date: Apr. 13, 2023

(54) Interface Component for a Flow Therapy Device

(71) Applicant: Masimo Corporation, Irvine, CA (US)

(72) Inventor: Omar Ahmed, Mission Viejo, CA (US)

(21) Appl. No.: 17/938,612

(22) Filed: Oct. 6, 2022

Related U.S. Application Data
(60) Provisional application No. 63/253,314, filed on Oct. 7, 2021.
(60) Provisional application No. 63/253,375, filed on Oct. 7, 2021.

(51) Int. Cl. A61M 16/06; A61B 5/1455; A61B 5/00; A61M 16/00

(52) U.S. Cl. CPC A61M 16/0672; A61B 5/14551; A61B 5/7475; A61M 16/024; A61M 2205/3368; A61M 2205/3334

(57) ABSTRACT

Various interface components for flow therapy devices are described herein. In some implementations, an interface component can include a first housing component that has a first upper surface and a second housing component that can include a second upper surface. The second housing component can be mounted on the first housing component such that the second upper surface is positioned higher than and angled relative to the first upper surface. The interface component can include a user interface that can control the flow therapy device. The user interface can be disposed on the second upper surface to enhance viewing and/or access.

### RELATED APPLICATIONS

This application claims the benefit of priority under 35 USC 119(e) to U.S. Provisional Application No. 63/253,314, filed Oct. 7, 2021, entitled “Interface Component for a Flow Therapy Device,” and to U.S. Provisional Application No. 63/253,375, filed Oct. 7, 2021, entitled “Interface Component for a Flow Therapy Device,” which are hereby incorporated by reference in their entireties.

### TECHNICAL FIELD

The present disclosure relates to devices, methods, and/or systems for an interface component of a flow therapy device.

### BACKGROUND

Hospitals, nursing homes, and other patient care facilities utilize flow therapy devices for spontaneously breathing patients. The flow therapy devices provide respiratory support through a nasal cannula interface and/or mouth cannula interface.

### SUMMARY

Flow therapy devices are increasingly more adaptable to the specific needs of a patient and provide increased functionality. For example, flow therapy devices can provide a wide range of flow rates of respiratory gas; supplement with oxygen; humidify and/or warm respiratory gas; provide condensate-free delivery of consistent, high-velocity flow during inspiration and exhalation; filter respiratory gas with a bacteria/viral filter; auto-identify types of nasal and/or mouth cannula(s) and breathing circuit(s) to improve and simplify workflow through automated configuration(s); include one or more flow drivers(s); and/or include other features and/or perform other functions. The flow therapy devices can indicate (visually and/or audibly) flow rate and/or respiratory gas characteristics and include one or more user interfaces to view and/or control said characteristics. The flow therapy devices can detect one or more parameters of a patient (e.g., respiratory rate, heart rate, blood oxygen level, etc.), communicate with one or more devices that can detect one or more parameters of the patient, and/or display one or more parameters of the patient. Various implementations of the devices disclosed herein provide improved devices, methods, and systems for interfacing the flow therapy devices with other devices and/or users.

Disclosed herein is a flow therapy device. The device can include an interface component that can have a first upper surface and a second upper surface. The second upper surface can be angled relative to the first upper surface and include a user interface disposed therein.

In some implementations, the second upper surface can be higher than the first upper surface.

In some implementations, the interface component can include a first housing component and a second housing component. The first housing component can include the first upper surface and the second housing component can include the second upper surface.

In some implementations, the second housing component can be mounted on the first housing component.

In some implementations, the first upper surface can slope downward as the first upper surface extends away from the second housing component.

In some implementations, the first housing component can include a first peripheral wall and the second housing component can include a second peripheral wall, the first peripheral wall can define a first circular periphery and the second peripheral wall can define a second circular periphery.

In some implementations, the first circular periphery can be larger than the second circular periphery.

In some implementations, the first circular periphery and the second circular periphery can be centered about an axis.

In some implementations, the interface component can include a handle. The handle can be disposed in the first housing component.

In some implementations, the second upper surface can include a receiving region that can receive the user interface therein.

In some implementations, the first upper surface can include a recess that can receive the second housing component therein.

In some implementations, a channel can be disposed in the recess. The channel can receive the second peripheral wall of the second housing component.

In some implementations, the interface component can include one or more cable connection interfaces that can facilitate communication with another device.

In some implementations, the second upper surface can be oriented at an acute angle relative to the first upper surface.

In some implementations, the second housing component can include an internal cavity housing a bracket that can facilitate coupling the second housing component to the first housing component such that the second upper surface is angled relative to the first upper surface.

In some implementations, the bracket can be angled in a longitudinal direction.

In some implementations, the bracket can be Y-shaped.

In some implementations, the first housing component can include a plurality of flanges for structural support.

In some implementations, the interface component can include a speaker.

In some implementations, the second housing component can include a tongue that can extend at an angle relative to the second upper surface.

In some implementations, the first housing component can include one or more openings for cables or the like to be routed internally from the second housing component into the first housing component.

In some implementations, the user interface can include a display.

In some implementations, the user interface can include a touchscreen.

In some implementations, the user interface can include one or more buttons control the device.

Disclosed herein is an interface component for a flow therapy device. The interface component can include a first housing component that can have a first upper surface. The interface component can include a second housing component that can include a second upper surface. The second housing component can be mounted on the first housing component such that the second upper surface can be positioned higher than and angled relative to the first upper surface. The interface component can include a user interface that can control the flow therapy device. The user interface can be disposed on the second upper surface.

In some implementations, the second housing component can be mounted on an upper surface of the flow therapy device.

In some implementations, the first upper surface can be angled relative to the upper surface of the flow therapy device.

In some implementations, the first housing component can include a gap in a periphery thereof that is aligned with an air flow connection region of the flow therapy device to facilitate connecting with a connector of a cannula.

Disclosed herein is an interface component of a flow therapy device may comprise a communication module configured to communicate with one or more physiological sensors and one or more computing devices. The interface component may include a processor in communication with the communication module and configured to receive data from the one or more physiological sensors or the one or more computing devices via the communication module. The processor may be configured to generate user interface data for rendering user interfaces, based at least in part, on the received data; and generate an alarm signal based at least in part, on the received data. The interface component may include a display in communication with the processor. The display may be configured to receive the generated user interface data from the processor and display one or more interactive graphical user interfaces according to the user interface data received from the processor. The interface component may include a speaker in communication with the processor and configured to receive the generated alarm signal from the processor; and output one or more auditory signals, according to the alarm signal.

In some implementations, the one or more physiological sensors may comprise a pulse oximeter or a temperature sensor, and wherein the one or more computing devices may comprise a mobile phone or laptop.

In some implementations, the data received by the processor via the communication module may comprise physiological parameters.

In some implementations, the data received by the processor via the communication module may comprise user commands from the one or more computing devices to control an operation of the interface component or the flow therapy device.

In some implementations, the processor may be configured to alter an operation of the interface component or the flow therapy device based, at in part, on data received from the one or more computing devices via the communication module.

In some implementations, the one or more physiological sensors and one or more computing devices may be remote to the interface component and the communication module may be configured to communicate wirelessly with the one or more physiological sensors and one or more computing devices.

In some implementations, the communication module may be configured to communicate with the one or more physiological sensors and the one or more computing devices via wires or cables.

In some implementations, the processor may be configured to calculate a ROX parameter based, at least in part, on data received from the one or more physiological sensors via the communication module.

In some implementations, the display may be configured to receive a user input to control an operation of the interface component or the flow therapy device.

For purposes of summarizing the disclosure, certain aspects, advantages, and novel features are discussed herein. It is to be understood that not necessarily all such aspects, advantages, or features will be embodied in any particular embodiment of the disclosure, and an artisan would recognize from the disclosure herein a myriad of combinations of such aspects, advantages, or features.

### BRIEF DESCRIPTION OF THE DRAWINGS

Certain features of this disclosure are described below with reference to the drawings. The illustrated embodiments are intended to illustrate, but not to limit, the embodiments. Various features of the different disclosed embodiments can be combined to form further embodiments, which are part of this disclosure.

FIG. 1A illustrates an interface component of a flow therapy device/apparatus in accordance with aspects of this disclosure.

FIG. 1B illustrates the interface component of the flow therapy device/apparatus of FIG. 1A with a cannula coupled thereto in accordance with aspects of this disclosure.

FIG. 2A illustrates the interface component of FIG. 1A in accordance with aspects of this disclosure.

FIG. 2B illustrates another view of the interface component of FIG. 1A in accordance with aspects of this disclosure.

FIG. 2C illustrates another view of the interface component of FIG. 1A in accordance with aspects of this disclosure.

FIG. 3A illustrates a partially exploded view of the interface component of FIG. 1A in accordance with aspects of this disclosure.

FIG. 3B illustrates another view of the partially exploded view of the interface component of FIG. 1A in accordance with aspects of this disclosure.

FIG. 4 illustrates another view of the interface component of FIG. 1A in accordance with aspects of this disclosure.

FIG. 5A is a schematic block diagram illustrating a monitoring system of a flow therapy device and interactive component.

FIG. 5B is a block diagram illustrating a controller of an interface component.

FIGS. 6A-6B, 7A-7D, 8A-8B, 9A-9D, and 10A-10C illustrate example graphical user interfaces that can be displayed by a display of the interface component.

FIGS. 11A-11D, 12A-12H, 13A-13I, 14A-14K, and 15A-15F illustrate example graphical user interfaces that can be displayed by a display of the interface component.

### DETAILED DESCRIPTION

Various features and advantages of this disclosure will now be described with reference to the accompanying figures. The following description is merely illustrative in nature and is in no way intended to limit the disclosure, its application, or uses. This disclosure extends beyond the specifically disclosed embodiments and/or uses and obvious modifications and equivalents thereof. Thus, it is intended that the scope of this disclosure should not be limited by any particular embodiments described below. The features of the illustrated embodiments can be modified, combined, removed, and/or substituted as will be apparent to those of ordinary skill in the art upon consideration of the principles disclosed herein.

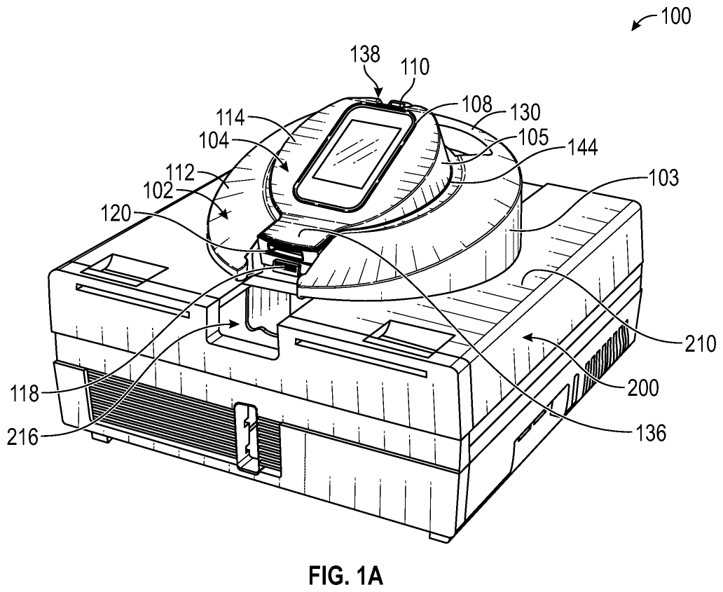

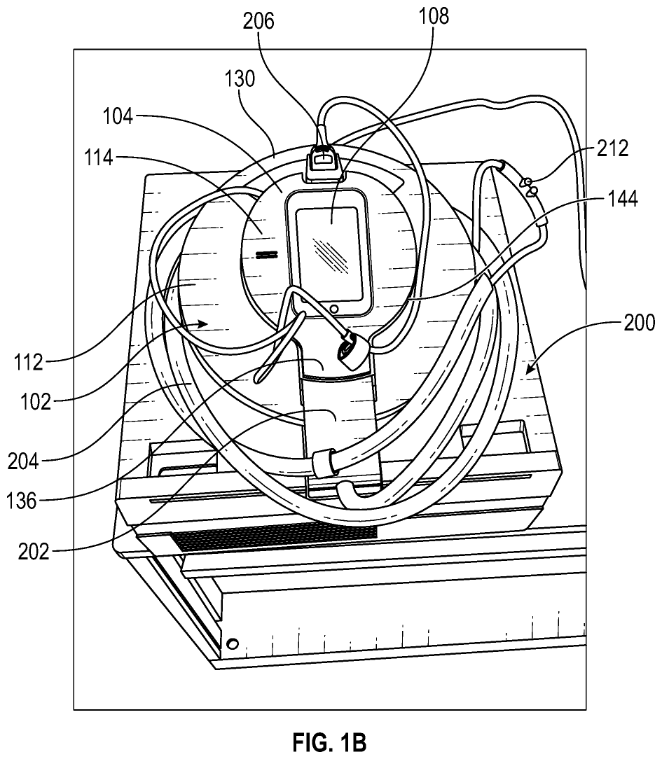

FIG. 1A illustrates a flow therapy device/apparatus 200 with an interface component 100 coupled thereto. The interface component 100 and flow therapy device/apparatus 200 can cooperate to provide respiratory gas to a patient. As described herein, the interface component 100 and flow therapy device/apparatus 200 can alter one or more characteristics of the provided respiratory gas (e.g., flow rate, oxygen level, etc.) to tailor to the specific needs of a patient and/or detect one or more parameters of the patient, which can include breathing parameters of the patient. In some implementations, the interface component 100 and flow therapy device/apparatus 200 can be in communication with one or more devices that can alter one or more characteristics of the respiratory gas (e.g., flow rate, oxygen level, etc.) to tailor to the specific needs of the patient and/or detect one or more parameters of the patient.

The interface component 100 can include a first housing component 102 and/or a second housing component 104. The first housing component 102 can be mounted on the flow therapy device/apparatus 200, which can include being mounted on an upper surface 210 of the flow therapy device/apparatus 200. The second housing component 104 can be mounted on the first housing component 102 such that the first housing component 102 is disposed between the flow therapy device/apparatus 200 and the second housing component 104. The first housing component 102 can include a receiving region 144 (e.g., recess), which can be disposed in a first upper surface 112 of the first housing component 102, that can receive at least a portion of the second housing component 104 therein. In some implementations, the first housing component 102 and second housing component 104 can be formed together, being one component.

The first housing component 102 can include the first upper surface 112. The first upper surface 112 can be disposed at an angle (e.g., acute angle) relative to the upper surface 210 of the flow therapy device/apparatus 200. The angle between the first upper surface 112 and the upper surface 210 of the flow therapy device/apparatus 200 can be less than forty-five, forty-five, or greater than forty-five degrees. The upper surface 210 of the flow therapy device/apparatus 200 can be disposed parallel relative to a surface supporting the flow therapy device/apparatus 200. The first upper surface 112 can be disposed at an angle (e.g., acute) relative to the surface supporting the flow therapy device/apparatus 200. In some implementations, the upper surface 210 of the flow therapy device/apparatus 200 can be disposed at an angle relative to the surface supporting the flow therapy device/apparatus 200. In some implementations, the first upper surface 112 can be disposed parallel to the upper surface 210 of the flow therapy device/apparatus 200.

The second housing component 104 can include a second upper surface 114. The second upper surface 114 can be disposed at an angle (e.g., acute) relative to the first upper surface 112 of the first housing component 102. The second upper surface 114 can be disposed at an angle (e.g., acute) relative to the upper surface 210 of the flow therapy device/apparatus 200. The second upper surface 114 can be disposed at an angle (e.g., acute) relative to the surface supporting the flow therapy device/apparatus 200. The angle between the second upper surface 114 and the first upper surface 112 of the first housing component 102, upper surface 210 of the flow therapy device/apparatus 200, and/or surface supporting the flow therapy device/apparatus 200 can be less than forty-five, forty-five, greater than forty-five, and/or ninety degrees. In some implementations, the second upper surface 114 can be parallel to the first upper surface 112 of the first housing component 102. The first upper surface 112 can be angled or sloped downward away from the receiving region 144 and/or second housing component 104

The second housing component 104 can house a user interface 108, which can include a display (e.g., touchscreen) and one or more buttons or the like to control the flow therapy device/apparatus 200. The user interface 108 can be disposed in the second upper surface 114 of the second housing component 104. The user interface 108 can display one or more characteristics of the provided respiratory gas (e.g., flow rate, oxygen content, etc.) and enable a user to manipulate the one or more characteristics to tailor to the specific needs of a patient. The user interface 108 can display one or more parameters of the patient, such as respiratory characteristics of the patient. The angling of the second upper surface 114 relative to the first upper surface 112 of the first housing component 102, upper surface 210 of the flow therapy device/apparatus 200, and/or surface supporting the flow therapy device/apparatus 200 and/or the angling of the first upper surface 112 relative to the upper surface 210 of the flow therapy device/apparatus 200 and/or surface supporting the flow therapy device/apparatus 200 can position the user interface 108 to enable a user to conveniently view the user interface 108 and/or interact with the one or more buttons or the like. The user interface 108 can be fixedly mounted to the second housing component 104. In some implementations, the user interface 108 can be removed from the second housing component 104 and communicate (wired or wirelessly) with the flow therapy device/apparatus 200. In some implementations, the user interface 108 can be a portable electronic device, such as a tablet.

The relative angling of the second upper surface 114 of the second housing component 104 and first upper surface 112 of the first housing component 102 can be fixed. In some implementations, the second housing component 104 can be tilted relative to the first housing component 102 to change the angle of the second upper surface 114 of the second housing component 104 relative to the first upper surface 112 of the first housing component 102, which can enable a user to manipulate the position of the user interface 108 for improved viewing and/or access. In some implementations, the second housing component 104 can include a handle 136 that can be manipulated by a user to change the angle of the second upper surface 114 of the second housing component 104 relative to the first upper surface 112 and/or flow therapy device/apparatus 200. In some implementations, the angle of the user interface 108 can be altered to facilitate enhanced viewing and/or access. In some implementations, the handle 136 can extend away from the second upper surface 114 at an angle such that the handle 136 is parallel to an upper surface 210 of the flow therapy device/apparatus 200 and/or a surface supporting the flow therapy device/apparatus 200.

The first housing component 102 can include a handle or grasping portion 130. The handle 130 can be disposed at a periphery of the first housing component 102. The handle 130 can follow a curved periphery of the first housing component 102. The handle 130 can be disposed opposite an air flow connection region 116 of the flow therapy device/apparatus 200.

The first housing component 102 can include a first peripheral wall 103 that can define a periphery of the first housing component 102. The first peripheral wall 103 can define a shape of the periphery, which can at least include circular, oval, polygonal (e.g., square, rectangle), irregular, and/or others. The first peripheral wall 103 can have a variable height, which can include having a larger height on one side compared to a shorter height on another side to facilitate the angled orientation of the first upper surface 112 relative to the upper surface 210 of the flow therapy device/apparatus 200. The first housing component 102 can include an internal cavity, which can be enclosed by at least the first peripheral wall 103 and first upper surface 112. The internal cavity can house one or more items therein.

The second housing component 104 can include a second peripheral wall 105 that can define a periphery 106 of the second housing component 104. The second peripheral wall 105 can define a shape of the periphery 106, which can at least include circular, oval, polygonal (e.g., square, rectangle), irregular, and/or others. The second peripheral wall 105 can have a variable height, which can include having a larger height on one side compared to a shorter height on another side to facilitate the angled orientation of the second upper surface 114 relative to the first upper surface 112 of the first housing component 102. The second housing component 104 can include an internal cavity, which can be enclosed by at least the second peripheral wall 105 and the second upper surface 114. The internal cavity can house one or more items therein.

The second peripheral wall 105 can be disposed radially inward relative to the first peripheral wall 103 of the first housing component 102. The second peripheral wall 105 of the second housing component 104 and the first peripheral wall 103 of the first housing component 102 can each define a circular periphery centered around the same axis.

The interface component 100 can interface with one or more devices and/or users. As described herein, the interface component 100 can include the user interface 108. The interface component 100 can include a cable connection interface 110 that can facilitate connecting to a cable or the like that can communicate data regarding the patient and/or treatment (e.g., one or more characteristics of the provided respiratory gas) and/or provide power to the flow therapy device/apparatus 200. The cable connection interface 110 can be disposed on the second housing component 104. The cable connection interface 110 can be disposed in a recess 138 formed in the second peripheral wall 105 and/or second upper surface 114. The cable connection interface 110 patient can be disposed on a side of the second housing component 104 with a longer second peripheral wall 105.

The flow therapy device/apparatus 200 can include an air flow connection region 216, which can also be described as an interface, that can facilitate connection with a cannula. For example, a connector of a cannula can be received in the air flow connection region 216 and connected to a connection interface 118, which can be disposed in the air flow connection region 216 of the flow therapy device/apparatus 200. The first housing component 102 can include an opening 120 through which the connector of the cannula can connect with a feature, which can include an internal feature, of the interface component 100. In some implementations, the air flow connection region 216 can be disposed at a front of the flow therapy device/apparatus 200.

FIG. 1B illustrates a connector 202 of a cannula 204 that can deliver respiratory gases to a patient disposed in the air flow connection region 216. As described herein, the connector 202 can couple to the connection interface 118 and/or to another component of the interface component 100 through the opening 120. The cannula 204 can include a nasal interface 212 that can interface with the nares of the patient to deliver respiratory gases.

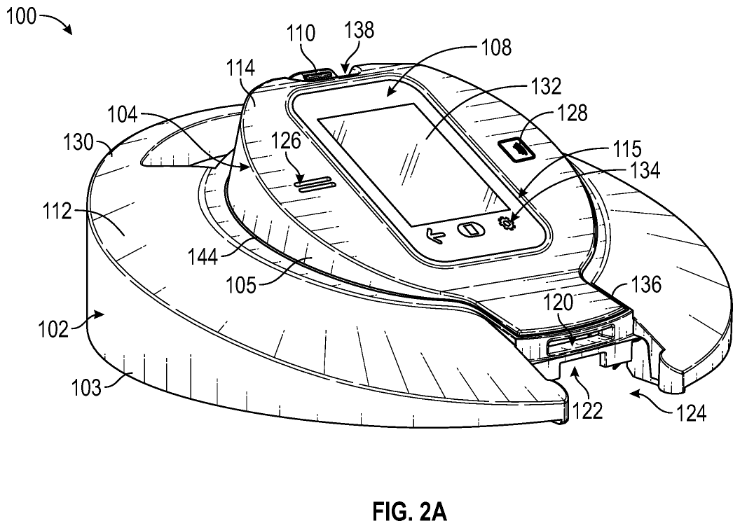

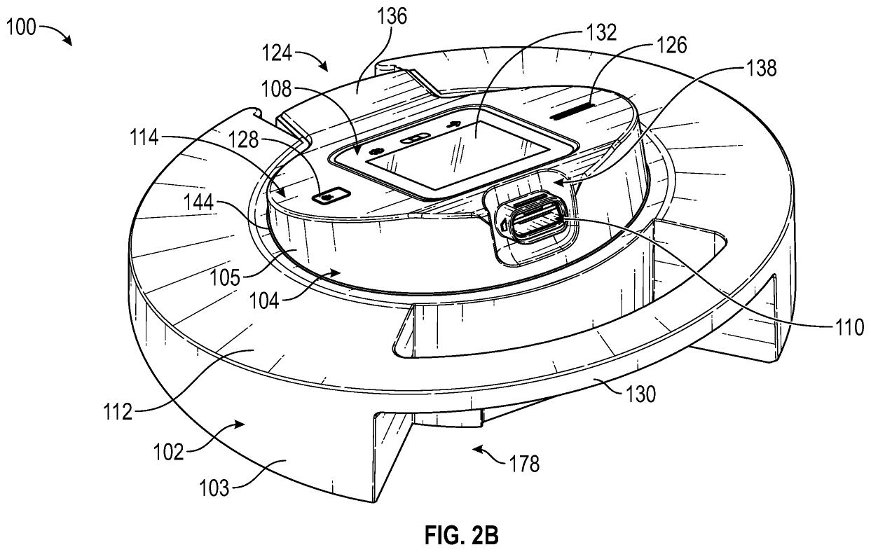

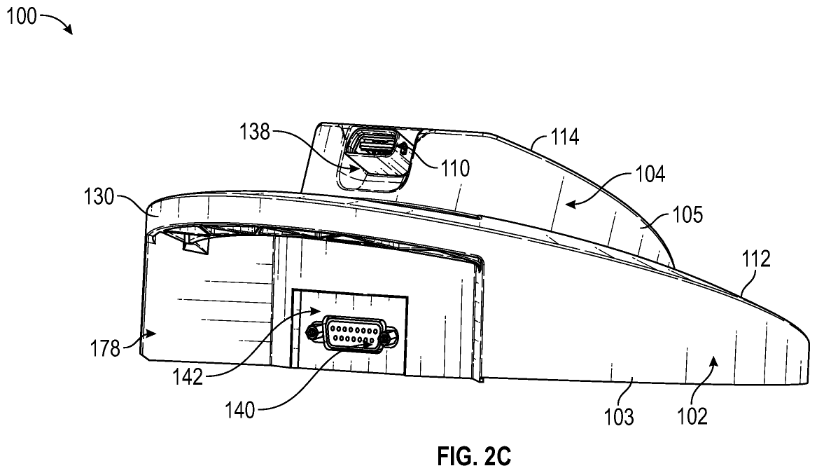

FIG. 2A-2C illustrate various views of the interface component 100. As illustrated in FIG. 2A, the second housing component 104 can include a receiving region 115, which can also be referred to as a recess, that can house the user interface 108 therein. The user interface 108 can include a display 132, which can be a touchscreen. The display 132 can indicate parameters of treatment and/or the patient and/or receive instructions from the user. The display 132 can display visual data and/or features through which the user can control the flow therapy device/apparatus 200 and/or view information. The user interface 108 can include one or more buttons 134 or the like, which can be virtual or mechanical, by which the user can navigate through visual data and/or features displayed to the user and/or control the flow therapy device/apparatus 200. The buttons 134 can at least include a back button, settings button, and/or home button.

The second housing component 104 can include a speaker 126, which can also include a microphone. The speaker 126 can audibly indicate information to the user regarding one or more parameters of treatment and/or the patient. In some implementations, the speaker 126 can sound an alarm to indicate a possible emergency (e.g., cannula obstruction, respiratory abnormality, etc.). In some implementations, the speaker/microphone 126 can receive audible commands from the user to control the flow therapy device/apparatus 200. The speaker 126 can project through holes in the second upper surface 114.

The second housing component 104 can include a near-field communication (NFC) symbol 128 to indicate that the flow therapy device/apparatus 200 can communicate utilizing NFC communication. The NFC symbol 128 can be disposed on the second upper surface 114.

The interface component 100 can include a space 178 disposed below the handle 130, as illustrated in FIG. 2B. The space 178 can disrupt the generally circular periphery of the first housing component 102. The space 178 can enable the user to grasp the handle 130. The space 178 can enable the user to access a cable connection interface 140 illustrated in FIG. 2C. The cable connection interface 140 can facilitate connecting the interface component 100 to a cable or the like. The cable connection interface 140 can be disposed in a recess 142 in the first peripheral wall 103 of the first housing component 102.

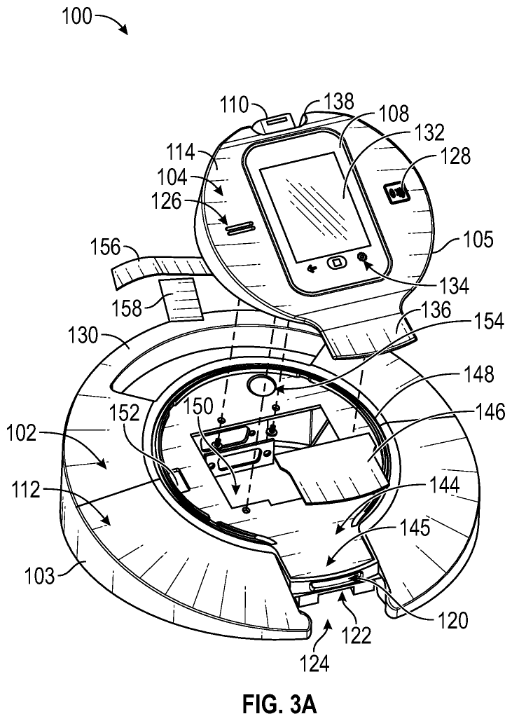

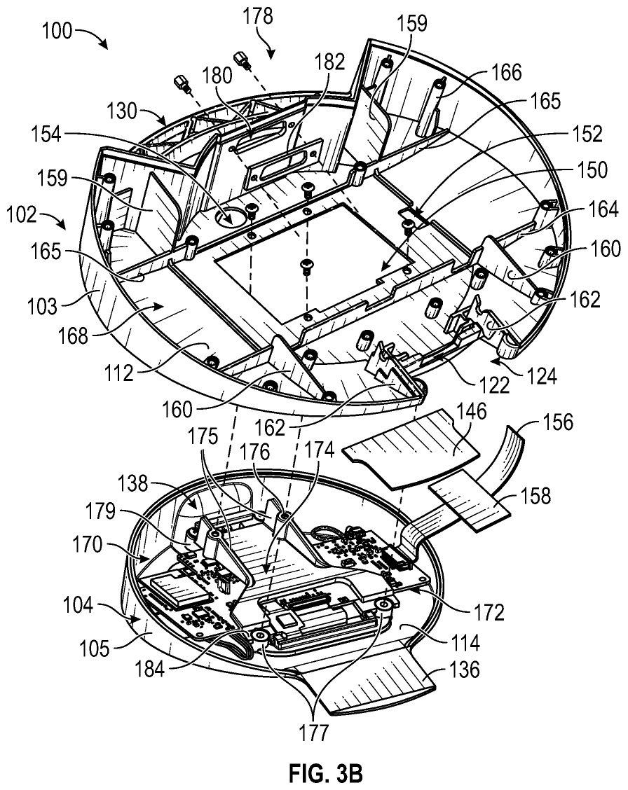

FIGS. 3A and 3B illustrate partially exploded views of the interface component 100 with the second housing component 104 decoupled from the first housing component 102. As illustrated in FIG. 3A, the first housing component 102 can include a receiving region 144 that can receive the second housing component 104. The receiving region 144 can be a recess in the first upper surface 112 of the first housing component 102. The receiving region 144 can be circular with a portion 145 extending therefrom to receive the handle or tongue 136 of the second housing component 104. The receiving region 144 can include a channel 148. The channel 148 can extend in a circular shape proximate a periphery of the receiving region 144. The channel 148 can receive the second peripheral wall 105 of the second housing component 104 therein when the second housing component 104 is disposed in the receiving region 144. The first upper surface 112 can be angled or curved downward as the first upper surface 112 extends away from the receiving region 144. In some implementations, the receiving region 144 can be planar while the surrounding first upper surface 112 can be curved or sloped downward as the first upper surface 112 extends away from the receiving region 144.

The first housing component 102 can include one or more openings to facilitate features (e.g., cables) housed within the internal cavity of the second housing component 104 to be routed into the internal cavity of the first housing component 102. The one or more openings can be disposed in the receiving region 144 of the first housing component 102. The one or more openings can include a main opening 150. The main opening 150 can be a variety of sizes and shapes, which can at least include circular, oval, polygonal (e.g., square, rectangle), irregular, and/or others. The one or more openings can include an aperture 154 that can be a variety of sizes and shapes, which can at least include circular, oval, polygonal (e.g., square, rectangle), irregular, and/or others. The one or more openings can include an aperture 152 through which a cable 156 (e.g., ribbon cable) can be routed from the second housing component 104 to the internal cavity of the first housing component 102. Tape 158 can be used to secure the cable 156 to an inside surface of the first housing component 102 with the cable 156 disposed through the aperture 152. The aperture 152 can be a variety of sizes and shapes, which can at least include circular, oval, polygonal (e.g., square, rectangle), irregular, and/or others.

The interface component 100 can include a release liner 146 that can be removed prior to coupling the first housing component 102 and second housing component 104 together.

As illustrated in FIG. 3B, the first housing component 102 can include an internal cavity 168. The cavity 168 can house one or more items therein. The cavity 168 can be at least partially defined by the first peripheral wall 103 and first upper surface 112 of the first housing component 102. The first housing component 102 can include one or more flanges that can provide structural support to the first housing component 102. The one or more flanges can be varying sizes and shapes to accommodate the angle of the first housing component 102.

For example, the first housing component 102 can include flanges 165, which can include two flanges 165. The flanges 165 can be disposed on an internal surface of the first upper surface 112. The flanges 165 can extend perpendicularly away from the internal surface of the first upper surface 112. The flanges 165 can be disposed on opposing sides of the main opening 150. The flanges 165 can extend away from the internal surface of the first peripheral wall 103. The flanges 165 can have a varied height along the longitudinal length thereof to accommodate for the sloping and/or angled portion of the first upper surface 112 that extends away from the receiving region 144.

The first housing component 102 can include flanges 159, which can include two flanges 159. The flanges 159 can extend away from an internal surface of the first peripheral wall 103 to respectively connect with portions of the flanges 165. The flanges 159 can be perpendicularly oriented relative to the flanges 165, respectively. The flanges 159 can extend away from the portion of the first peripheral wall 103 defining the space 178. The flanges 159 can be parallel to each other. The flanges 159 can be disposed on opposing sides of an aperture 180, which can be varying shapes and sizes, disposed in the first peripheral wall 103 that receives the cable connection interface 140. A frame 182 can be disposed around the aperture 180 and coupled to an internal surface of the first peripheral wall 103. The flanges 159 can have a varied height along the longitudinal length thereof to accommodate for the sloping and/or angled portion of the first upper surface 112 that extends away from the receiving region 144.

The first housing component 102 can include a flange 164. The flange 164 can include recesses or cutouts. The flange 164 can be disposed on and extend away from the internal surface of the first upper surface 112. The flange 164 can include a varied height along the longitudinal length thereof to accommodate for the sloping and/or angled portion of the first upper surface 112 that extends away from the receiving region 144. The flange 164 can extend between proximate opposing internal surfaces of the first peripheral wall 103. The flange 164 can be disposed on an opposing side of the main opening 150 relative to the flanges 165. The flange 164 can be parallel to the flanges 165. The flange 164 can extend from screw/bolt interfaces 166 extending from the inside surface of the first upper surface 112. The screw/bolt interfaces 166 can be projections with threaded internal cavities that facilitates coupling the first housing component 102 to the flow therapy device/apparatus 200 with bolts, screws, or the like.

The first housing component 102 can include flanges 160, which can include two flanges 160. The flanges 160 can be disposed on and extend away from the internal surface of the first upper surface 112. The flanges 160 can extend away from screw/bolt interfaces 166 extending from the inside surface of the first upper surface 112. The flanges 160 can be perpendicularly oriented relative to the flange 164. The flanges 160 can be aligned with the flanges 159. The flanges 160 can be disposed on opposing sides of a gap 124.

The first housing component 102 can include flanges 162, which can include two flanges 162. The flanges 162 can be disposed on opposing sides of a gap 122. The flanges 162 can extend away from the internal surface of the first upper surface 112. The flanges 162 can extend away from the internal surface of the first peripheral wall 103. The flanges 162 can be disposed between the flanges 160.

The second housing component 104 can include an internal cavity 170. The cavity 170 can house one or more items therein. The cavity 170 can be at least partially defined by the second peripheral wall 105 and the second upper surface 114 of the second housing component 104.

The second housing component 104 can include an electronic board 172 disposed in the cavity 170. The electronic board 172 can be connected to the cable connection interface 110 (FIG. 3A). The electronic board 172 can be connected to the cable 156 that can connect to one or more features in the cavity 168 of the first housing component 102 and/or flow therapy device/apparatus 200. The electronic board 172 can include the hardware to operate the user interface 108 and/or one or more features of the flow therapy device/apparatus 200.

The second housing component 104 can include a bracket 174. The bracket 174 can be generally Y-shaped. The bracket 174 can be angled in a longitudinal direction to accommodate for the angle of the second upper surface 114. The bracket 174 can be disposed over the electronic board 172 and couple to features disposed on an internal surface of the second upper surface 114. The bracket 174 can include screw/bolt interfaces 179 that facilitate coupling the bracket 174 to screw/bolt interfaces disposed on the internal surface of the second upper surface 114. The bracket 174 can include pronged portions 184, which can include two pronged portions 184. The pronged portions 184 can be disposed on an opposing side of the bracket 174 relative to the screw/bolt interfaces 179. The pronged portions 184 can be disposed on arm portions of the bracket 174. The pronged portions 184 can be positioned around screw/bolt interfaces 177 disposed on the internal surface of the second upper surface 114, such that the pronged portions 184 are secured in place when the first housing component 102 is coupled to the second housing component 104 at the screw/bolt interfaces 177. The bracket 174 can include flanges 175. The flanges 175 can include screw/bolt interfaces 176 that can facilitate coupling the bracket 174 to the first housing component 102 by way of screws, bolts, or the like. The flanges 175 can be varying thicknesses in a longitudinal direction to accommodate for the angle of the bracket 174. The screw/bolt interfaces 176 can be disposed at positions on the flanges 175 such that the screw/bolt interface 176 and screw/bolt interface 177 extend to a common plane, facilitating the second housing component 104 to be connected to the receiving region 144 of the first housing component 102. The receiving region 144 may, in some implementations, be planar.

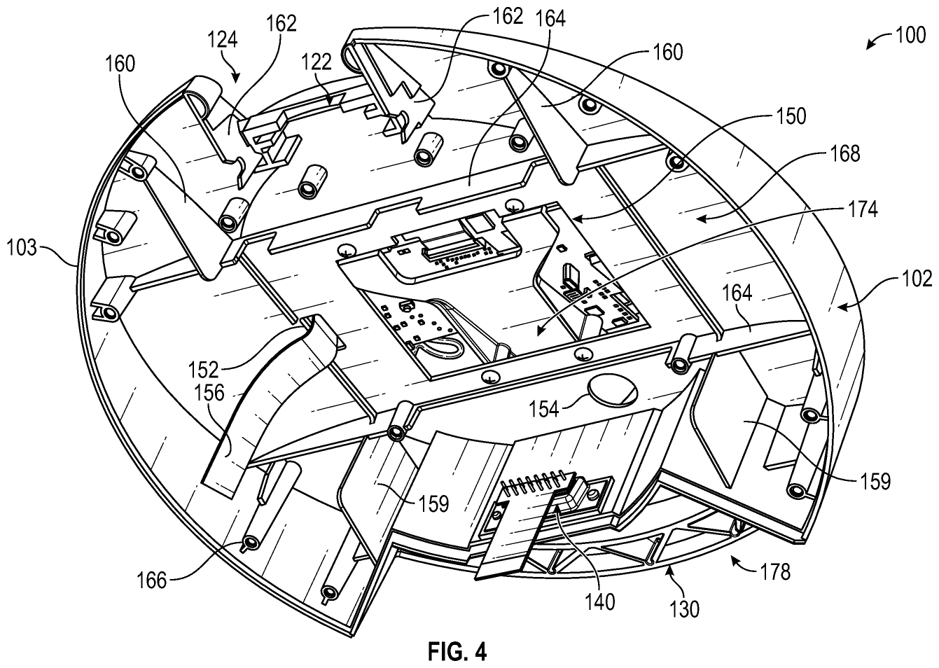

FIG. 4 illustrates the cavity 168 of the first housing component 102 with the first housing component 102 and second housing component 104 coupled together. As shown, the bracket 174 is coupled to the first housing component 102 such that the bracket 174 extends over the main opening 150. The cable 156 is disposed through the aperture 152 and into the cavity 168 of the first housing component 102.

In some implementations, the flow therapy device/apparatus 200, which can include the interface component 100, can be wirelessly paired with any of the sensors and/or devices shown in at least FIGS. 2A, 2D, 2E, 2F, and 2G of US Patent Application Publication No. 2021/0290184, titled “Remote Patient Management and Monitoring Systems and Methods,” filed Mar. 19, 2021, and published on Sep. 23, 2021, which is hereby incorporated by reference in its entirety. In some implementations, the therapy parameters of the flow therapy device/apparatus 200 can be remotely monitored and controlled. The interface component 100 can include one or more hardware processors that can enable signal processing for a determination of one or more physiological parameters. For example, signal processing can include analyzing plethysmograph signals and determination of oxygen saturation, pulse rate, and other related parameters. The one or more hardware processors can also connect with communications interface. The communications interface can enable serial communication and network communications, such as Wi-Fi and Bluetooth. Through the communications interface, the apparatus 200 can connect with remote monitoring systems described in detail in the above-incorporated US Patent Application Publication No. 2021/0290184.

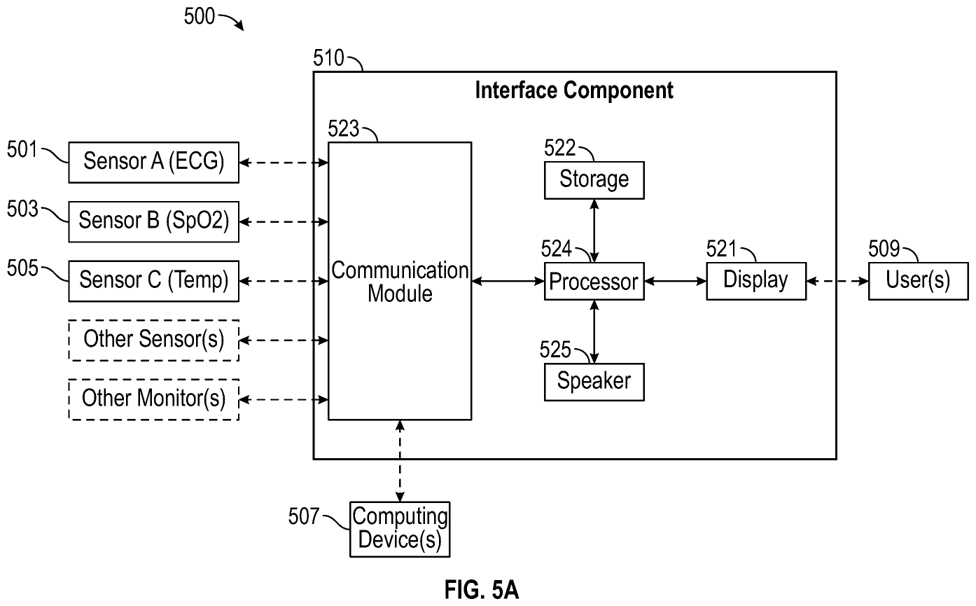

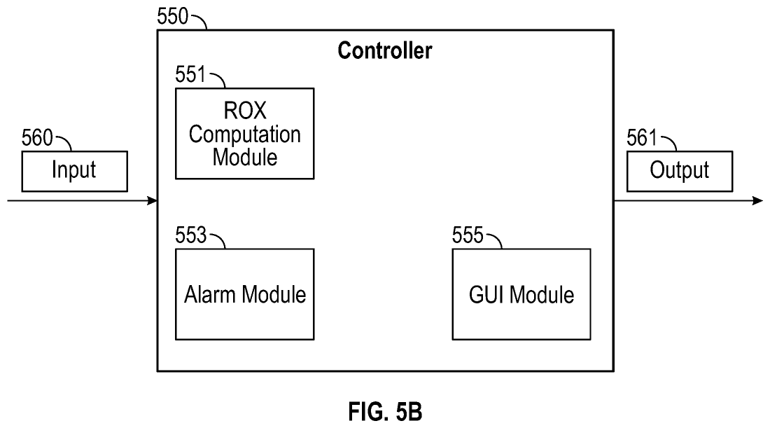

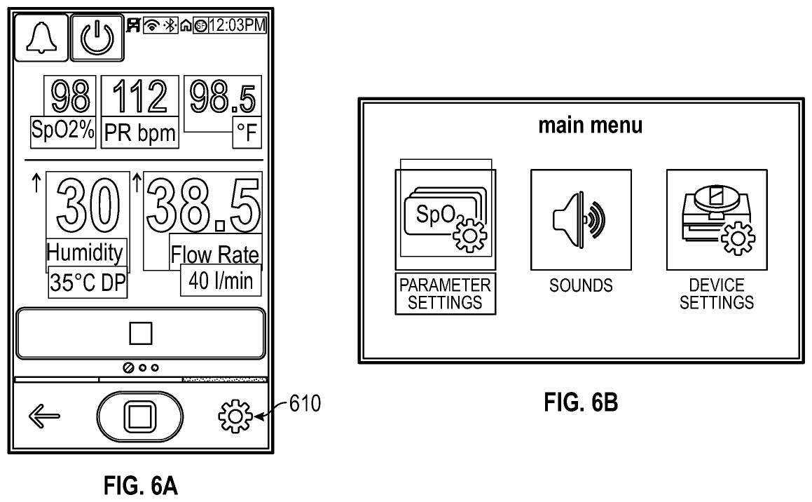

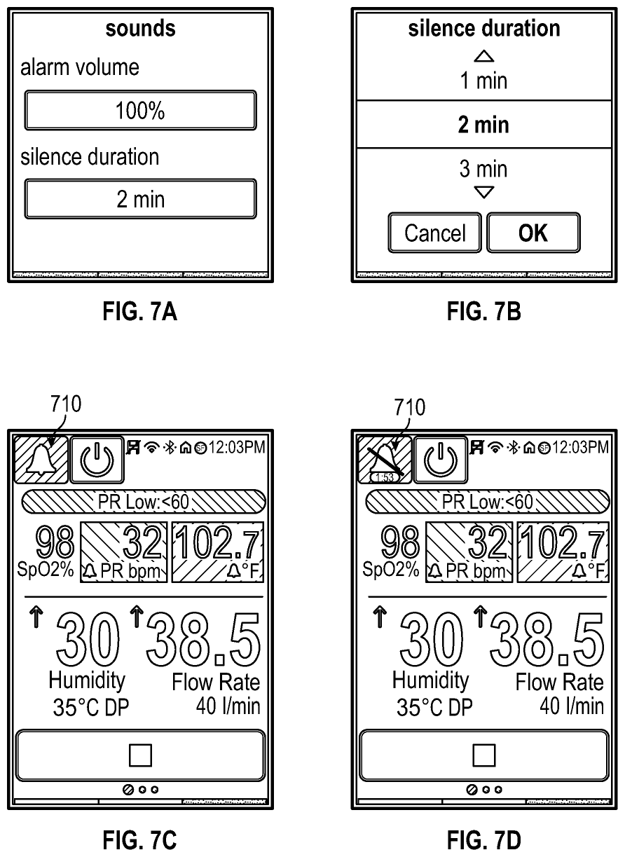

FIG. 5A illustrates a block diagram of a system architecture including a network interface as discussed above. FIG. 5B illustrates software engines that can be implemented by the one or more hardware processors. FIGS. 6A-6B, 7A-7D. 8A-8B, 9A-9D, 10A-10C, 11A-11B, 12A-12H, 13A-13I, 14A-14K, and 15A-15F illustrate examples of graphical user interfaces displaying one or more physiological parameters and alarm conditions determined by one or more hardware processor. The graphical interfaces can be displayed on the display 132 of FIG. 3A.

FIG. 5A is a schematic block diagram illustrating a monitoring system 500. The monitoring system 500 can include an interface component 510 in communication with various sensors, monitors or users. For example, the interface component 510 is illustrated in communication with an ECG sensor 501, an SpO2 sensor 503 and a temperature sensor 505. The interface component 510 may be part of a flow therapy device, such as flow therapy device 100 shown in FIGS. 1A-4. The interface component 510 may include similar operational and/or structural features as discussed with reference to FIGS. 1A-4.

As shown in FIG. 5A, the interface component 510 can include a communication module 523, a processor 524, a storage 522, a display 521, and a speaker 525. The storage device 522 can include one or more memory devices that store data, including without limitation, dynamic and/or static random access memory (RAM), programmable read-only memory (PROM), erasable programmable read-only memory (EPROM), electrically erasable programmable read-only memory (EEPROM), and the like. Such stored data can be processed and/or unprocessed physiological data obtained from external sensors or monitors or user commands or instructions or settings, for example.

The communication module 523 can facilitate communication (via wired and/or wireless connection) between external devices or components (such as physiological sensors or monitors) or users and/or computing devices and the interface component 510. For example, the communication module 523 can be configured to allow the interface component 510 to wirelessly communicate with other devices, systems, and/or networks over any of a variety of communication protocols. The communication module 523 can be configured to use any of a variety of wireless communication protocols, such as Wi-Fi (802.11x), Bluetooth®, ZigBee®, Z-wave®, cellular telephony, infrared, near-field communications (NFC), RFID, satellite transmission, proprietary protocols, combinations of the same, and the like. The communication module 523 can allow data and/or instructions to be transmitted and/or received to and/or from the interface component 510 and separate external devices. The communication module 523 can be configured to receive (for example, wirelessly) processed and/or unprocessed physiological data or other information such as user commands or instructions from separate computing devices, which can include, among others, a mobile device (for example, an iOS or Android enabled smartphone, tablet, laptop), a mobile phone, a desktop computer, a server or other computing or processing device for display and/or further processing, among other things. The communication module 523 can be embodied in one or more components that are in communication with each other. The communication module 523 can comprise a wireless transceiver, an antenna, and/or a near field communication (NFC) component, for example, an NFC transponder.

As shown in FIG. 5A, the communication module 523 may be in communication (e.g., wirelessly or wired) with external devices such as physiological sensors such as an ECG sensor, an SpO2 sensor, a temperature sensor, and the like. The communication module 523 may be in communication with any type of sensor as required or desired and/or with other physiological monitors. The communication module 523 may be configured to receive unprocessed physiological data, such as raw signals, from the physiological sensors or monitors. The communication module 523 may be configured to receive processed physiological data, such as physiological parameters, from the physiological sensors or monitors.

The communication module 523 may be in communication (e.g., wirelessly or wired) with one or more computing devices 507. The communication module 523 may be configured to receive data from the computing device(s) 507, which may include user commands or instructions which may control an operation or functionality of the interface component 510. For example, a user may adjust the settings of the interface component 510 via the computing device(s) 507 which may be remote to the interface component 510. In some implementations, the interface component 510 may transmit data to the computing device(s) 507 for the computing device to render interactive user interfaces similar to those discussed and shown herein such that the computing device(s) 507 may display user interfaces similar to those displayed by the display 521 such that a user may monitor the flow therapy, patient, operation of the flow therapy device etc. remotely to the flow therapy device and/or patient. Advantageously, a user (such as a care provider) may control operation of the interface component 510 remotely which may facilitate a reduced contact or exposure of the care provider to a patient with which the interface component 510 may be in close proximity.

The interface component 510 can include a processor 524. The processor 524 may be in communication with the communication module 523 and may also be in communication with the display 521 and the speaker 525. The processor 524 may include more than one processor. The processor 524 can be configured, among other things, to process data, execute instructions to perform one or more functions, and/or control the operation of the interface component 510. For example, the processor 524 can process physiological data, signals, parameters etc. obtained from external sensors, monitors, computing devices or commands from a user and can execute instructions to perform functions related to such physiological data or user commands such as generating alarms, generating graphical user interface data to render a user interface on a display and the like. The processor 524 can be configured to generate data for rendering graphical user interfaces displaying information representative of the processed or unprocessed physiological data and/or other information obtained from the communication module 523. The processor 524 can be configured to generate a signal to cause the speaker 525 to output an audio signal such as an alarm.

The interface component 510 can include a display 521. The display 521 may be in communication with the processor 524. The display 521 may be an interactive display such as a capacitive touchscreen. The display 521 may be configured to display interactive graphical user interfaces. The display 521 may be configured to receive user input (such as via a touch on the screen by a user 509). The processor 524 may be configured to generate and/or execute instructions according to input from the user 509 via the display 521.

The interface component 510 can include the speaker 525. The speaker 525 may be in communication with the processor 524. The speaker 525 may be configured to generate and output auditory signals such as alarms. The speaker 525 may be configured to output one or more different types of auditory signals according to instructions received from the processor 524.

FIG. 5B is a block diagram illustrating a controller 550 of an interface component. The controller 550 may be included as part of the processor 524 shown in FIG. 2A. The controller 550 may include an ROX computation module 551, an alarm module 553, and a GUI module 555. The controller 550 may be configured to receive an input 560 and generate an output 561. The input 560 may include information received from a communication module and/or from a user via a display, as discussed elsewhere herein. The output 561 may include one or more signals such as to a display or speaker to generate an interactive user interface to display on the display or to generate an audio signal to output at the speaker. The ROX computation module 551 may be configured to calculate ROX, as described in greater detail herein. The alarm module 553 may be configured to determine when to generate an alarm and what type of alarm to generate, as described in greater detail herein. The GUI module 555 may determine the data for rendering graphical user interfaces and may receive and process user input received via an interactive display.

ROX Calculation

ROX may be defined as the ratio of oxygen saturation (SpO2) (as measured by pulse oximetry/FiO2) to respiratory rate. The controller 550 may be configured to receive physiological parameters such as SpO2, FiO2 or respiration rate, for example from external sensors as discussed with reference to FIG. 5A. In some embodiments, the controller 550 may be configured to receive FiO2 data and/or respiratory rate data from the flow therapy device in which it is integrated. The controller 550 may be configured to calculate ROX for example according to the following formula: (SpO2/FiO2)/respiration rate. The controller may be configured to output the calculated ROX, for example to a display.

### Example Alarm Settings

FIGS. 6A-6B, 7A-7D, 8A-8B, 9A-9D, and 10A-10C illustrate example interactive graphical user interfaces that can be displayed by the display of the interface component of the flow therapy device discussed herein.

The user interface shown in FIG. 6A may be displayed on the display and a user may select a component 610 which may navigate the user to the user interface shown in FIG. 6B. The user interface shown in FIG. 6B may be a main menu with selectable components that a user can select to control operation on the interface component such as parameter settings, sounds, device settings etc.

The user interface shown FIG. 7A may allow a user to adjust settings relating to alarms and sounds of the interface component. For example, a user may select a silence duration for an alarm as shown in FIGS. 7A and 7B. The silence duration may a timeframe during which an alarm is silent after a user has silence the alarm. For example, the user interface of FIG. 7C shows an alarm that has been triggered (for example in response to a low patient pulse rate and/or high patient temperature). A user may select the selectable icon 710 shown in FIG. 7C to silence the alarm. After a user has selected the icon 710, the alarm may be silenced as shown in FIG. 7D. The alarm may be silent for the silence duration according to the settings, for example, as selected by a user as discussed and shown with reference to FIGS. 7A and 7B. Upon expiration of the silence duration (e.g., 2 minutes) the alarm may again begin to sound.

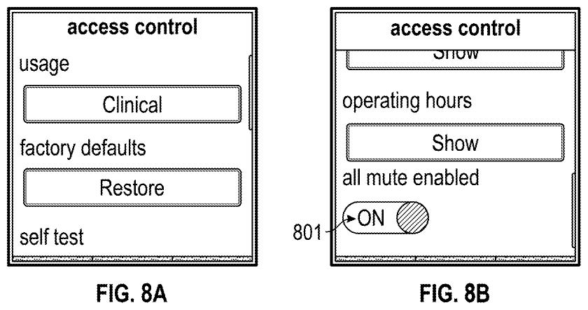

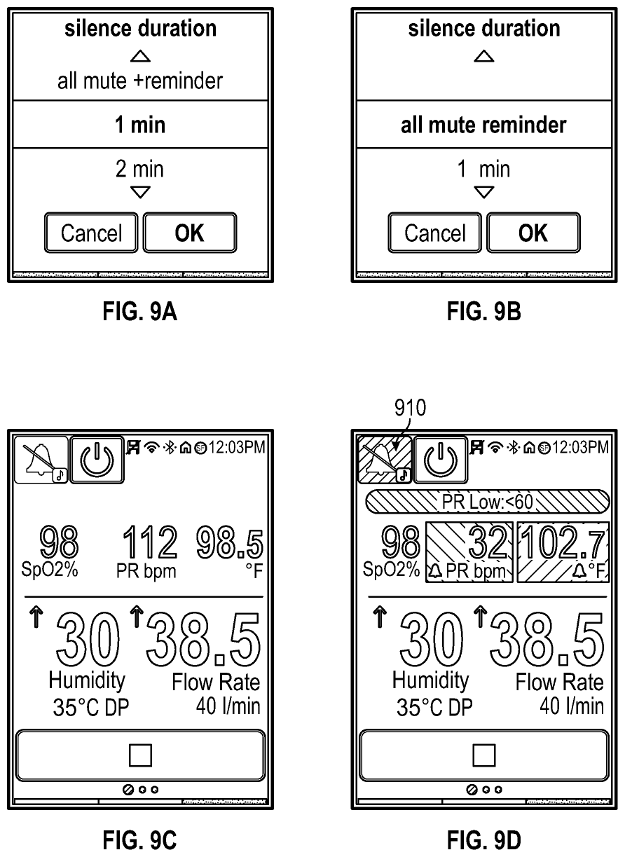

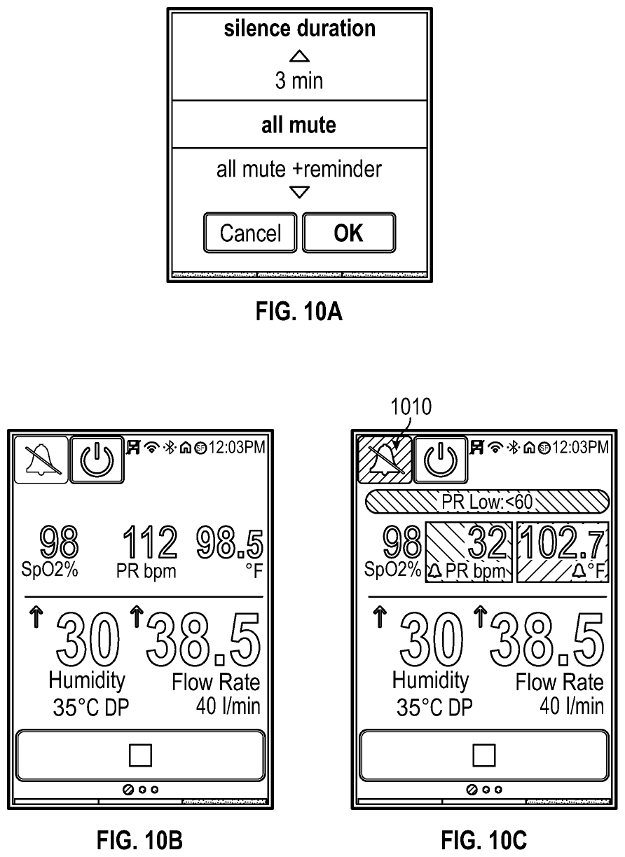

FIGS. 8A and 8B are additional user interfaces for adjusting settings relating to an alarm of the interface component. As shown in FIG. 8B, a user may select to turn on muting all alarms or to turn off muting all alarms. The alarms may be muted with a reminder (as discussed and shown with reference to FIGS. 9A-9B) or the alarms may be muted without a reminder (as discussed and shown with reference to FIGS. 10A-10C).

FIGS. 9A-9D are example user interfaces illustrating settings when an all mute has been enabled (e.g., as shown in FIG. 8B). As shown in FIGS. 9A and 9B, a user may select a time frame for a reminder such as one minute. The user interface of FIG. 9D shows an alarm that has been triggered (for example in response to a low patient pulse rate and/or high patient temperature). The icon 910 shows that the alarm has been muted. A reminder may alarm upon expiration of the time frame set according to FIGS. 9A and 9B.

FIGS. 10A-10B are example user interfaces illustrating settings when an all mute has been enabled (e.g., as shown in FIG. 8B). As shown in FIG. A, a user can select to mute all alarms without a reminder. The user interface of FIG. 10C shows an alarm that has been triggered (for example in response to a low patient pulse rate and/or high patient temperature). The icon 1010 shows that the alarm has been muted. A reminder may not alarm because no reminder has been selected according to the settings selected in FIG. 10A.

### Example Graphical User Interfaces

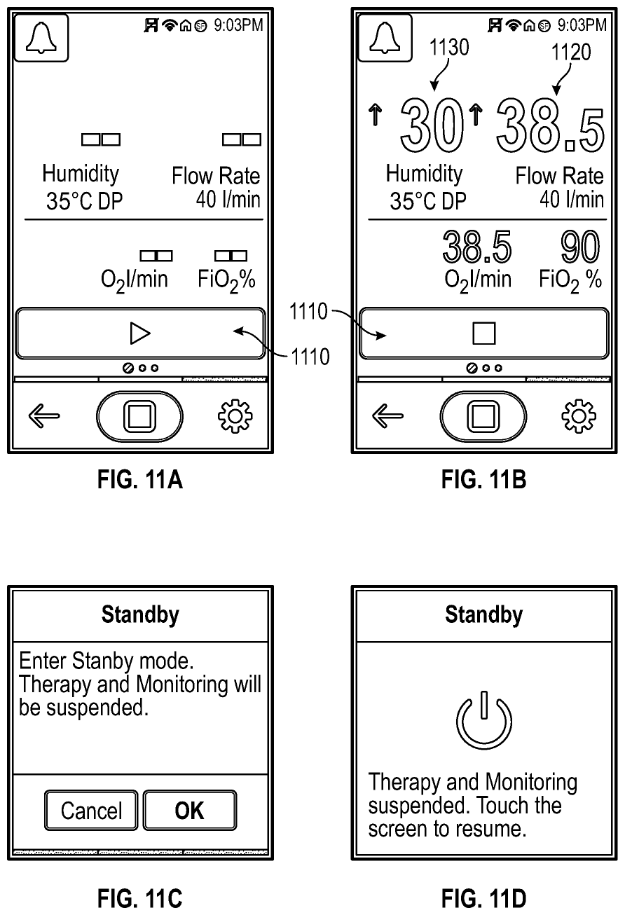

FIGS. 11A-11D, 12A-12H, 13A-13I, 14A-14K, and 15A-15F illustrate example interactive graphical user interfaces that can be displayed by the display of the interface component of the flow therapy device discussed herein.

FIG. 11A shows an example user interface when the flow therapy device has not yet initiated a flow therapy. A user may select the component 1110 to cause the flow therapy device to initiate a flow therapy.

FIG. 11B shows an example user interface when the flow therapy device is administering a flow therapy to a patient. The user interface can show information relating to humidity, flow rate, oxygen consumption rate, FiO2 etc. A user may select the component 1110 to stop or pause the flow therapy. A user may select components 1130 to adjust humidity of the flow therapy or may adjust component 1120 to adjust a flow rate of the flow therapy. FIGS. 11C and 11D show example user interfaces relating to a standby mode of the flow therapy device.

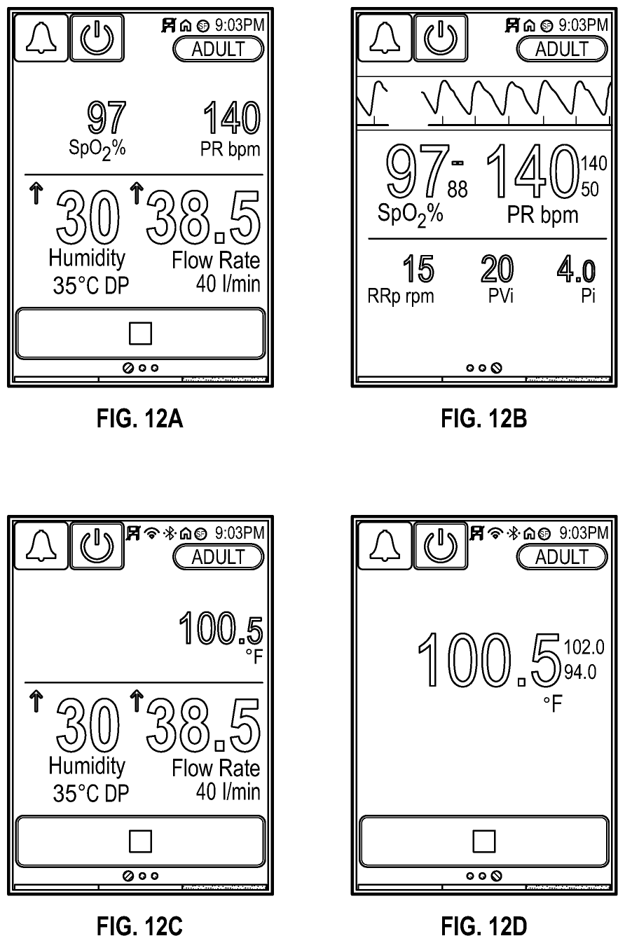

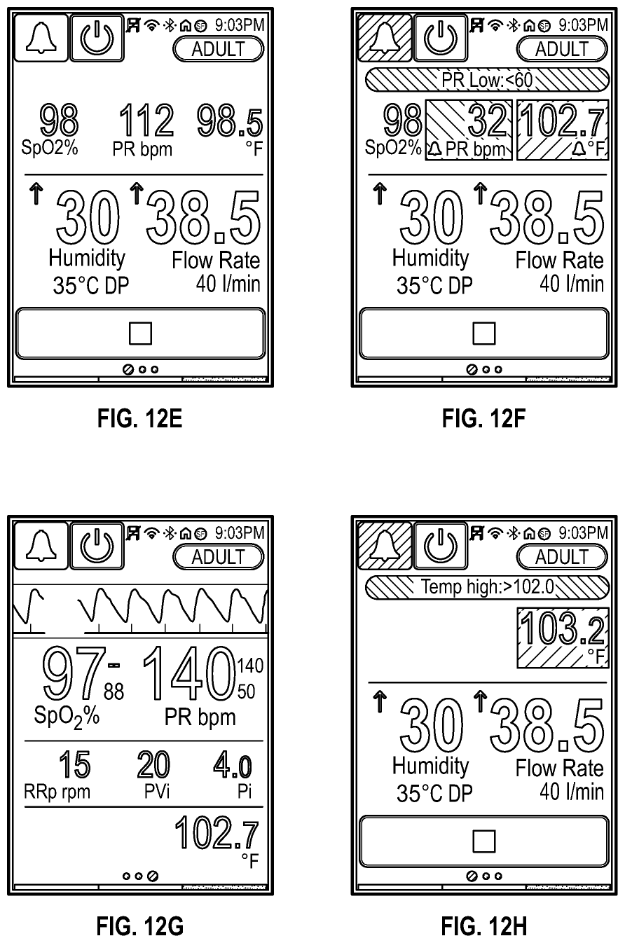

FIGS. 12A and 12B show example user interfaces displaying information relating to a flow therapy as well as information relating to a pulse oximetry of a patient (e.g., SpO2 and pulse rate). This information may be gathered from one or more sensors external to the flow therapy device, for example as discussed with reference to FIG. 5A. FIGS. 12C and 12D show example user interfaces displaying information relating to a flow therapy as well as information relating to a temperature of a patient. This information may be gathered from one or more sensors external to the flow therapy device, for example as discussed with reference to FIG. 5A. FIGS. 12E-12H show example user interfaces displaying information relating to a flow therapy, a pulse oximetry (e.g., SpO2 and pulse rate) and a temperature of a patient. As shown in FIGS. 12F and 12H, the interface component of the flow therapy device may generate one or more alarms in response to satisfaction of one or more conditions such as a pulse rate or SpO2 of the patient dropping below a threshold value and/or a temperature of the patient exceeding a threshold value.

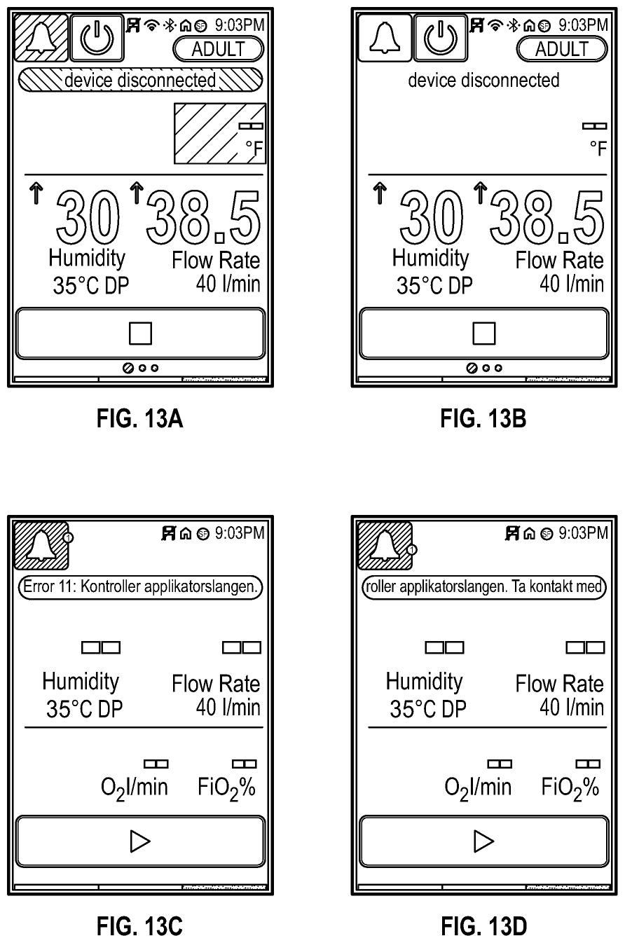

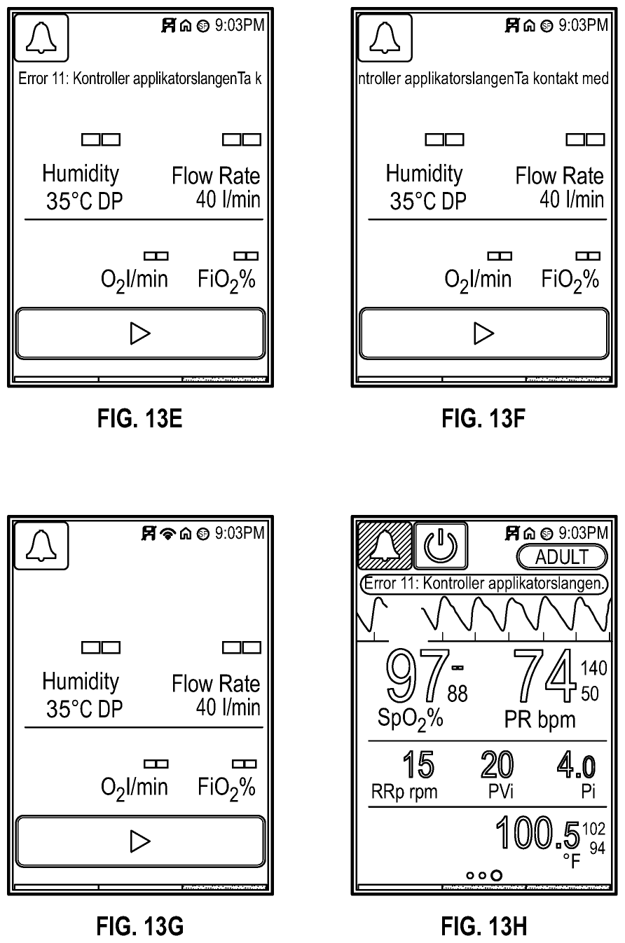

FIGS. 13A-13I show example user interfaces relating to alarms, error messages and notifications. In some embodiments, the error messages, notifications and/or alarms shown in FIGS. 13A-13I may disappear once they have been acknowledged by a user. FIG. 131 shows an example user interface that may be displayed in response to a duplicate sensor being connected to the flow therapy device. For example, if the flow therapy is already connected to an oximeter sensor via Bluetooth and a user attempts to connect the device to an oximeter device via wires or cables, the interface component may display the user interface shown in FIG. 13I. This user interface may also be shown if a user attempts to connect a sensor via wires or cables and a sensor is already connected via Bluetooth. This user interface may be shown for duplicates of any type of sensor.

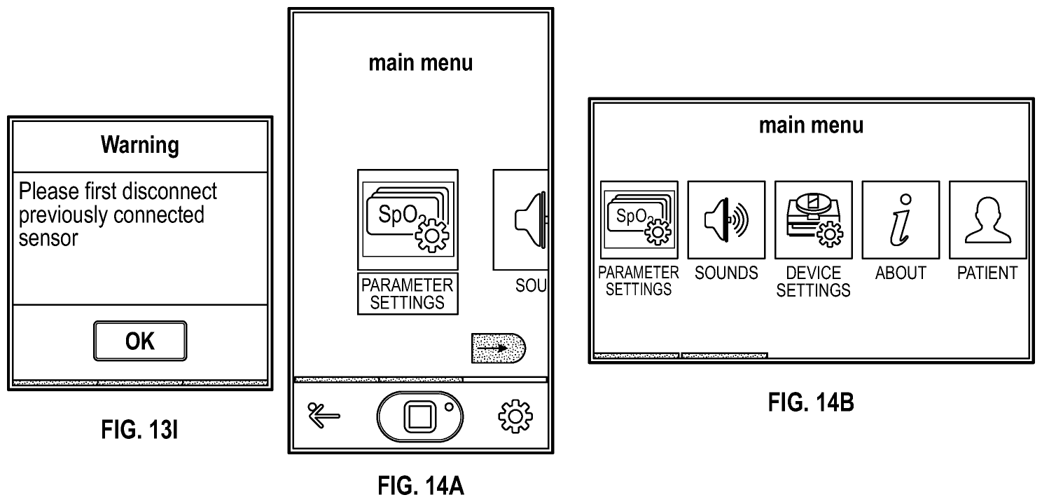

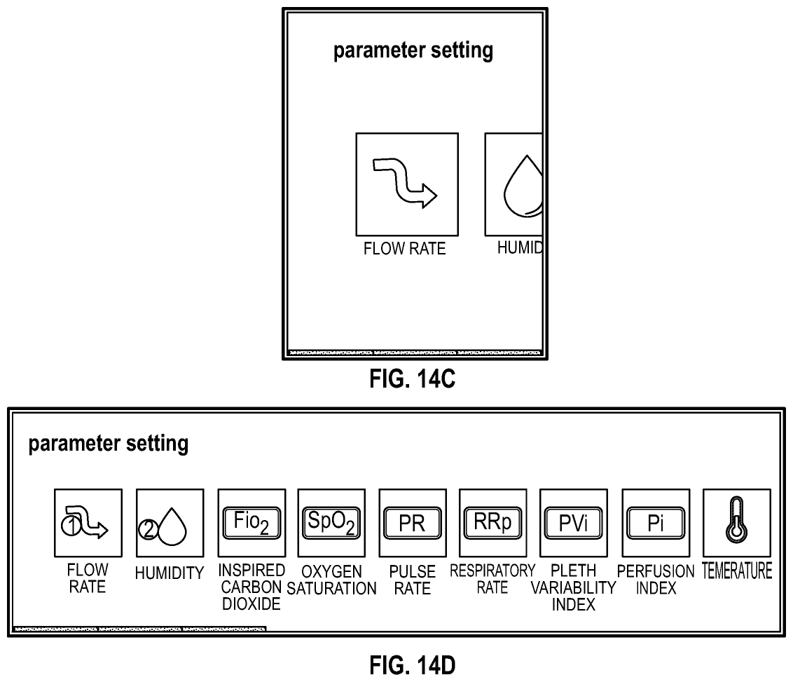

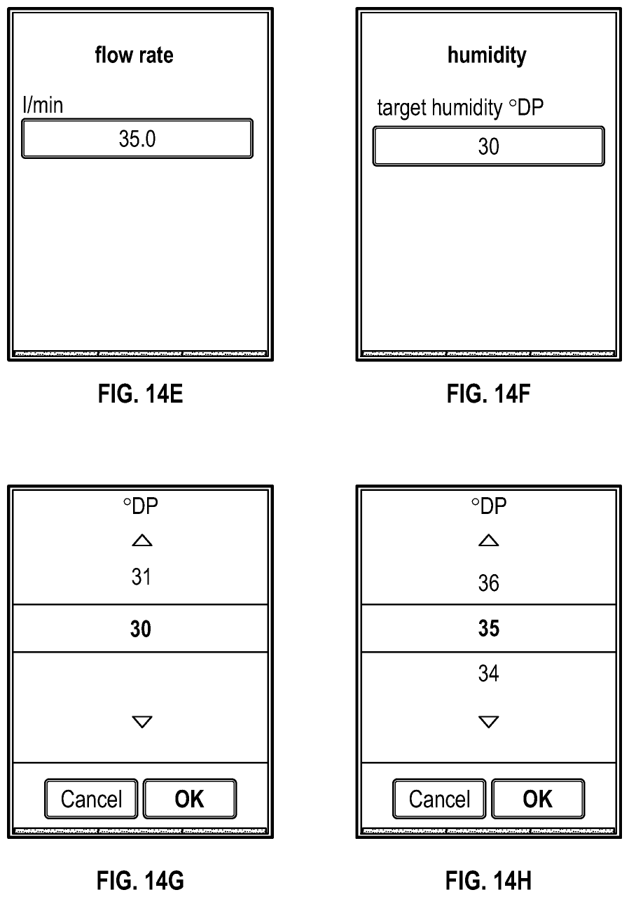

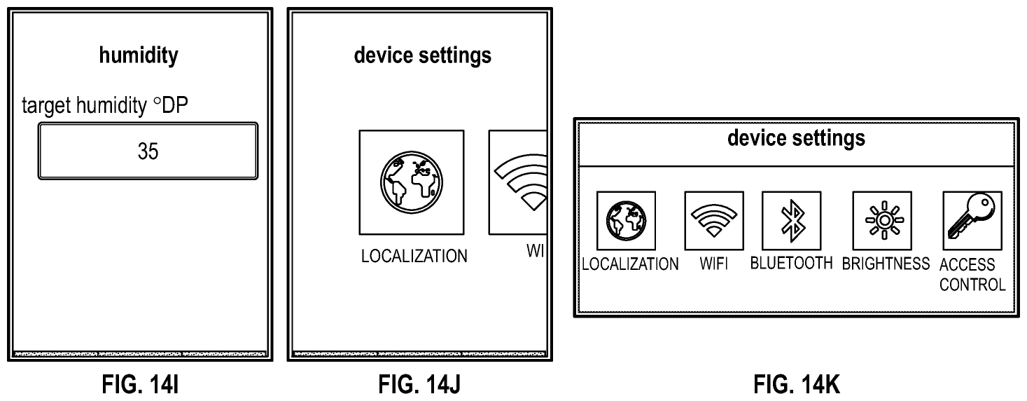

FIGS. 14A-14K show example user interfaces relating to menus for adjusting settings of the interface component. FIGS. 14A-14B show example user interfaces of a main menu. FIGS. 14C-14D show example user interfaces of a menu for adjusting parameter settings of a flow therapy device. FIGS. 14E-141 show example user interfaces for adjusting settings of a flow therapy such as flow rate or humidity. FIGS. 14J-14K show example user interfaces for adjusting settings of the flow therapy device.

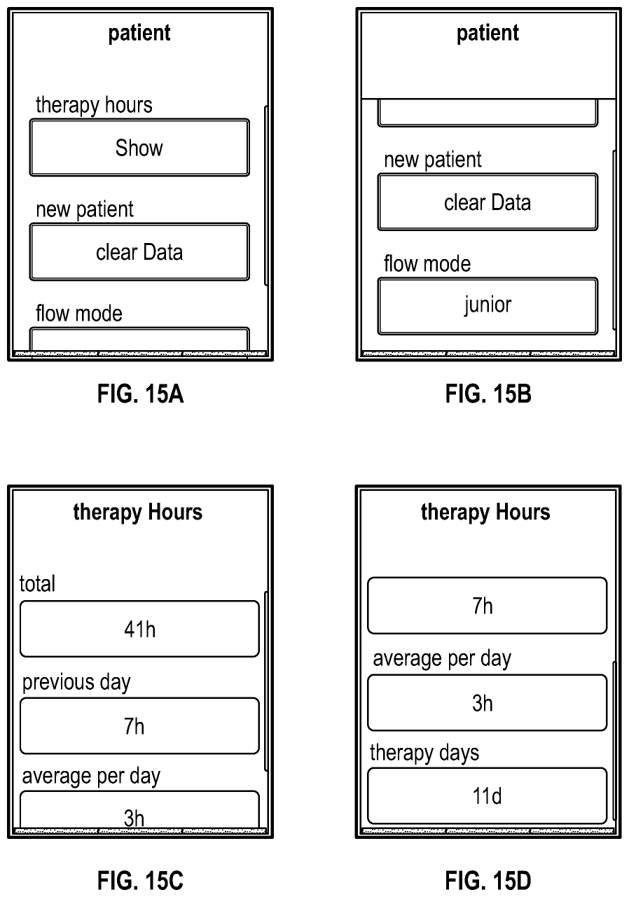

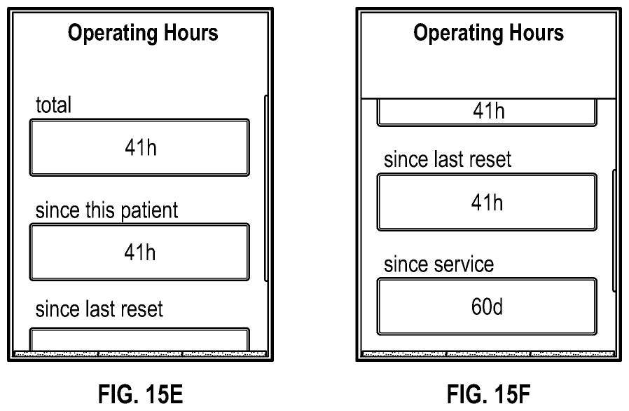

FIGS. 15A-15F show example user interfaces of patient menus for displaying and receiving information relating to a patient's flow therapy.

Additional Considerations and Terminology

Although this invention has been disclosed in the context of certain preferred embodiments, it should be understood that certain advantages, features and aspects of the systems, devices, and methods may be realized in a variety of other embodiments. Additionally, it is contemplated that various aspects and features described herein can be practiced separately, combined together, or substituted for one another, and that a variety of combination and subcombinations of the features and aspects can be made and still fall within the scope of the invention. Furthermore, the systems and devices described above need not include all of the modules and functions described in the preferred embodiments.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more embodiments necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree.

Although certain embodiments and examples have been described herein, it will be understood by those skilled in the art that many aspects of the systems and devices shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Any methods disclosed herein need not be performed in the order recited. The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (for example, not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

Various illustrative logical blocks, modules, routines, and algorithm steps that may be described in connection with the disclosure herein can be implemented as electronic hardware (e.g., ASICs or FPGA devices), computer software that runs on general purpose computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and modules that may be described in connection with the disclosure herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. A processor can include an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

As various changes could be made in the above constructions without departing from the scope of the invention, it is intended that all the matter contained in the above description or shown in the accompanying drawings shall be interpreted as illustrative and not in a limiting sense.

What is claimed is:

1. A flow therapy device, the device comprising:
an interface component comprising a first upper surface and a second upper surface, the second upper surface angled relative to the first upper surface and comprising a user interface disposed therein.

2. The device of claim 1, wherein the second upper surface is higher than the first upper surface.

3. The device of claim 1, wherein the interface component comprises a first housing component and a second housing component, the first housing component comprising the first upper surface and the second housing component comprising the second upper surface.

4. The device of claim 3, wherein the second housing component is mounted on the first housing component.

5. The device of claim 3, wherein the first upper surface slopes downward as the first upper surface extends away from the second housing component.

6. The device of claim 3, wherein the first housing component comprises a first peripheral wall and the second housing component comprises a second peripheral wall, the first peripheral wall defining a first circular periphery and the second peripheral wall defining a second circular periphery.

7. The device of claim 6, wherein the first circular periphery is larger than the second circular periphery.

8. The device of claim 6, wherein the first circular periphery and the second circular periphery are centered about an axis.

9. The device of claim 1, wherein the interface component comprises a handle.

10. The device of claim 9, wherein the handle is disposed in the first housing component.

11. The device of claim 3, wherein the second upper surface comprises a receiving region that is configured to receive the user interface therein.

12. The device of claim 11, wherein the first upper surface comprises a recess that is configured to receive the second housing component therein.

13. The device of claim 12, further comprising a channel disposed in the recess, the channel configured to receive the second peripheral wall of the second housing component.

14. The device of claim 1, wherein the interface component comprises one or more cable connection interfaces configured to facilitate communication with another device.

15. The device of claim 1, wherein the second upper surface is oriented at an acute angle relative to the first upper surface.

16. The device of claim 3, wherein the second housing component further comprises an internal cavity housing a bracket configured to facilitate coupling the second housing component to the first housing component such that the second upper surface is angled relative to the first upper surface.

17. The device of claim 16, wherein the bracket is angled in a longitudinal direction.

18. The device of claim 17, wherein the bracket is Y-shaped.

19. The device of claim 3, wherein the first housing component comprise a plurality of flanges for structural support.

20. The device of claim 1, wherein the interface component comprises a speaker.

21. The device of claim 3, wherein the second housing component comprises a tongue extending at an angle relative to the second upper surface.

22. The device of claim 3, wherein the first housing component comprises one or more openings for cables or the like to be routed internally from the second housing component into the first housing component.

23. The device of claim 1, wherein the user interface comprises a display.

24. The device of claim 1, wherein the user interface comprises a touchscreen.

25. The device of claim 1, wherein the user interface comprises one or more buttons to control the device.

26. An interface component for a flow therapy device, the interface component comprising:
a first housing component comprising a first upper surface;
a second housing component comprising a second upper surface, the second housing component mounted on the first housing component such that the second upper surface is positioned higher than and angled relative to the first upper surface; and
a user interface configured to control the flow therapy device, the user interface disposed on the second upper surface.

27. The interface component of claim 26, wherein the second housing component is mounted on an upper surface of the flow therapy device.

28. The interface component of claim 27, wherein the first upper surface is angled relative to the upper surface of the flow therapy device.

29. The interface component of claim 26, wherein the first housing component comprises a gap in a periphery thereof that is aligned with an air flow connection region of the flow therapy device to facilitate connecting with a connector of a cannula.
